(19) United States
(12) Patent Application Publication
(10) Pub. No.: US 2005/0014832 A1
(43) Pub. Date: Jan. 20, 2005

(54) Treatment

(75) Inventors: Marianne O'Shea, Channahon, IL (US); Ulrike Schmid, Wormerveer (NL); Ingrid Celestina Maria Mohede, Wormerveer (NL); Frederick William Cain, Wormerveer (NL)

Correspondence Address: MORGAN LEWIS & BOCKIUS LLP, 1111 PENNSYLVANIA AVENUE NW, WASHINGTON, DC, 20004, US

(73) Assignee: LC USA LLC

(21) Appl. No.: 10/619,549

(22) Filed: Jul. 16, 2003

(51) Int. Cl. A61K 31/202

(52) U.S. Cl. 514/560

(57) ABSTRACT

A method of preventing or treating a common cold in a mammal, or of treating or ameliorating the symptoms of a common cold in a mammal, comprises administering to said mammal an effective amount of conjugated fatty acid or a derivative thereof.

This invention relates to a method of preventing or treating a common cold. In particular, the invention relates to the use of certain compounds and their derivatives for this purpose.

The term “common cold” is a well-known term used by both medical practitioners and the public in general and refers to illnesses caused by a viral infection which is located in the nose, but which may also involve the sinuses, ears and bronchial tubes. The symptoms of the common cold include sneezing, a runny nose, nasal obstruction or stuffiness, sore or itchy throat, cough, hoarseness and mild general symptoms such as headache, fever, chilliness and a general feeling of being unwell. It is known that the common cold is not a single entity, but rather is a group of diseases caused by members of several families of viruses. More than 200 viruses are known to cause the symptoms of the common cold. The most important viruses are the coronaviruses, picornaviruses, rhinoviruses, coxackieviruses and adenoviruses. Other viruses associated with the common cold include parainfluenza viruses, respiratory syncytial viruses and enteroviruses.

Numerous methods of treating the common cold have been described. These include, for example, WO 02/09699 which describes a method of treating a common cold comprising administration of a flavonoid alone or in combination with a metal, and WO 02/40023 which discloses the use of an NK3 antagonist in the treatment of the common cold.

Although many remedies exist for the common cold and its symptoms, there remains a need for treatments of the common cold, and the symptoms of the common cold, that are relatively inexpensive, yet safe and effective.

U.S. Pat. No. 4,851,437 asserts that various tung oil compositions are useful for treating body deficiencies as varied as cancer, AIDS, ageing and schizophrenia, but fails to support these assertions with any data.

U.S. Pat. No. 5,674,901 and U.S. Pat. No. 5,827,885 disclose the use of conjugated linoleic acid (CLA) to maintain or elevate CD-4 and CD-8 cell levels to prevent or alleviate the adverse effects of TNF or a virus. Fifteen broad families of viruses are listed covering viruses as diverse as HIV and hepatitis B, but the only example given is the treatment of fowl pox virus. Neither rhinoviruses nor the treatment of the common cold is mentioned in either document.

According to the present invention in a first aspect, there is provided a method of preventing or treating a common cold in a mammal, or of treating or ameliorating the symptoms of a common cold in a mammal, which comprises administering to said mammal an effective amount of one or more substances selected from conjugated fatty acids and derivatives thereof.

In another aspect, the present invention provides the use of a conjugated fatty acid or a derivative thereof in the manufacture of a composition for the prevention or treatment of the common cold, or the treatment or amelioration of symptoms of the common cold in a mammal. The invention also contemplates a conjugated fatty acid or a derivative thereof for use in the prevention or treatment of the common cold, or the treatment or amelioration of symptoms of the common cold in a mammal.

The term “preventing or treating a common cold in a mammal, or of treating or ameliorating the symptoms of a common cold in a mammal”, includes prophylaxis and fill or partial treatment. It may also include reducing the symptoms of the common cold, ameliorating the symptoms of the common cold, reducing the severity of the common cold or its symptoms, reducing the incidence of the common cold, or any other change in the condition of the patient, which improves the therapeutic outcome. In a preferred aspect of the invention, the method or use of the invention has at least the effect of reducing the recovery time after a common cold.

Symptoms of the common cold include one or more of sneezing, a runny nose/rhinitis, nasal obstruction (blocked nose or stuffiness), sore or itchy throat, coughing, hoarseness, asthma exacerbation and mild general symptoms such as headache, fever, chilliness and a general feeling of being unwell (or malaise). The invention may therefore comprise the treatment or prevention of one or more of sneezing, runny nose, nasal obstruction, sore throat, itchy throat, coughing, hoarseness, asthma exacerbation, headache, fever, chilliness and malaise, especially sore throat.

The invention is particularly useful when the common cold is caused by a coronavirus or a rhinovirus. In particular, the present invention is directed to the treatment, including prophylaxis, of a viral infection in a human, which is caused by the human rhinovirus (HRV), or a coronavirus. The viruses include the various different serogroups.

Without wishing to be bound by theory, it is believed that the conjugated fatty acid may exert activity, at least in part, against viruses responsible for the common cold by influencing ICAM-1 (intercellular adhesion molecule-1, which is a receptor for certain viruses), and/or by affecting CD58 cell levels.

Mammals for treatment according to the invention are not limited and include, for example, cats, dogs, cattle, sheep and horses. Preferably, the mammal is a human.

In the invention, the mammal is typically administered a composition comprising the conjugated fatty acid or a derivative thereof. The conjugated fatty acid may be used in the form of the free acid. Derivatives of conjugated fatty acids include salts and esters thereof, or a mixture of two or more of these materials. Salts are non-toxic, pharmaceutically acceptable and/or acceptable for use in food products and/or pharmaceuticals and include, for example, salts with alkali metals and alkaline earth metals such as sodium, calcium and magnesium, preferably sodium. Esters include, for example, mono-, di- and tri-glycerides and mixtures thereof, and C₁ to C₆ alkyl esters (where the alkyl group can be straight chain or branched), as well as esters formed with alcohols that are acceptable in food products or pharmaceutical products, such as are disclosed in EP-A-1167340, the contents of which are incorporated by reference herein. Suitable alcohols include terpene alcohols or sesquiterpene alcohols, for example menthol, isopulegol, menthenol, carveol, carvomenthenol, carvomenthol, isobomylalcohol, caryophyllenealcohol, geraniol, famesol and citronellol.

Conjugated fatty acids may be diunsaturated (i.e., containing two carbon-carbon double bonds) or polyunsaturated (i.e, containing more than two carbon-carbon double bonds) and are compounds that contain at least a pair of adjacent carbon-carbon double bonds (e.g., one or more —CH═CH—CH═CH— linkages). Preferably the conjugated fatty acids are diunsaturated. The two carbon-carbon double bonds in the conjugated fatty acids may each be in a cis or trans configuration and conjugated fatty acids therefore exist in the form of a number of geometrical isomers. For use in the invention, the conjugated fatty acids may be pure isomers or mixtures of isomers. Conjugated fatty acids according to the invention are preferably straight chain carboxylic acids. Conjugated fatty acids according to the invention contain from 12 to 24 carbon atoms, preferably from 14 to 22 carbon atoms, more preferably from 16 to 20 carbon atoms, such as 18 carbon atoms (the number of carbon atoms refers to the carbon atom of the carboxylic acid group and the carbon atoms of the alkenyl chain attached to the carbon atom of the carboxylic acid group). Preferred conjugated fatty acids for the invention are conjugated linoleic acid (CLA) and conjugated linolenic acid, with CLA being particularly preferred.

Conjugated fatty acids and derivatives thereof may be used in the invention alone (i.e., as a single conjugated fatty acid) or as mixtures of two or more conjugated fatty acids or of two or more derivatives of conjugated fatty acids. Suitable mixtures also include mixtures of one or more conjugated fatty acids with one or more derivatives of the same or different conjugated fatty acids.

Conjugated linoleic acid (CLA), which is the preferred conjugated fatty acid for use in the present invention, may comprise one isomer or a mixture of two or more different isomers including: cis, cis; cis, tans; trans, cis; and trans, trans isomers. Preferred isomers are the trans10, cis12 and cis9, trans 11 isomers, including these isomers in relatively pure form, as well as mixtures with each other and/or mixtures with other isomers. More preferably, the conjugated linoleic acid or derivative thereof comprises trans10, cis12 and cis9, trans11 isomers and the weight ratio of trans10, cis12 isomer to cis9, trans11 isomer is at least 1.2:1, such as 1.3:1, even more preferably at least 1.5:1, e.g., in the range 1.5:1 to 100:1 or 1.5:1 to 10:1, such as a 60:40 or 80:20 mixture of the trans10, cis12: cis9, trans11 isomers. Particularly preferred are compositions comprising the trans10, cis 12 isomer as the major isomer component i.e., present in an amount of at least 55%, preferably at least 60%, more preferably at least 70%, even more preferably at least 75%, most preferably at least 80%, such as at least 90% or even 100% by weight based on the total amount of conjugated linoleic acid.

Conjugated fatty acids can be produced in conventional ways. For example, conjugated linoleic acid can be produced by klown methods, such as that described in EP-A-902082, the contents of which are incorporated herein by reference. Conjugated linoleic acid products that are enriched in one or more isomers are disclosed in WO 97/18320, the contents of which are also incorporated herein by reference.

The conjugated fatty acid is preferably used and/or administered in the form of a composition. Suitable compositions are, preferably, a pharmaceutical composition, a foodstuff or a food supplement. These compositions provide a convenient form in which to deliver the conjugated fatty acid. Compositions of the invention may comprise an antioxidant in an amount effective to increase the stability of the conjugated fatty acid or derivative thereof with respect to oxidation.

The amount of conjugated fatty acid or derivative thereof that is administered in the method of the invention or that is for administration in the use of the invention is preferably from about 0.1 g to about 20 g (more preferably 0.1 g to 10 g, such as 0.5 g to 5 g) of conjugated fatty acid or derivative thereof per day. Suitable compositions can be formulated accordingly.

A preferred composition according to the invention is a foodstuff. Food products (which term includes animal feed) contain a fat phase, wherein the fat phase contains the product of the invention. The foodstuffs are optionally used as a blend with a complementary fat. For example, the blend may comprise 0.3-95 wt %, preferably 2-80 wt %, most preferably 540 wt % of the product of the invention and 99.7-5 wt %, preferably 98-20 wt %, most preferably 95-60 wt % of a complementary fat selected from: cocoa butter, cocoa butter equivalents, palm oil or fractions thereof, palmkernel oil or fractions thereof, interesterified mixtures of said fats or fractions thereof, or liquid oils, selected from: sunflower oil, high oleic sunflower oil, soybean oil, rapeseed oil, cottonseed oil, fish oil, safflower oil, high oleic safflower oil, maize oil and MCT-oils. Examples of suitable foodstuffs include those selected from the group consisting of margarines, fat continuous or water continuous or bicontinuous spreads, fat reduced spreads, confectionery products such as chocolate or chocolate coatings or chocolate fillings or bakery fillings, ice creams, ice cream coatings, ice cream inclusions, dressings, mayonnaises, cheeses, cream alternatives, dry soups, drinks, cereal bars, sauces, snack bars, dairy products, clinical nutrition products and infant formulations.

Other examples of compositions are pharmaceutical compositions, such as in the form of tablets, pills, capsules, caplets, multiparticulates including: granules, beads, pellets and micro-encapsulated particles; powders, elixirs, syrups, suspensions and solutions. Pharmaceutical compositions will comprise a pharmaceutically acceptable diluent or carrier. Pharmaceutical compositions are preferably adapted for administration parenterally (e.g., orally). Orally administrable compositions may optionally comprise one or more of a decongestant, an anti-histamine and an anti-pyretic (e.g., paracetamol). Orally administrable compositions may be in solid or liquid form and may take the form of tablets, powders, suspensions and syrups. Optionally, the compositions comprise one or more flavouring and/or colouring agents. Pharmaceutical compositions may be formulated in other ways, such as for administration to a mucosa, including a nasal mucosa; such compositions may optionally comprise at least one humectant. Humectants are capable of absorbing or retaining water and include, for example, mineral oils, vegetable oils, soothing agents, cellulose derivatives, sugars, alcohols, polymers, or membrane conditioners, in particular glycerol, sorbitol, propylene glycol, glycerine, and polyethylene glycols.

Pharmaceutically acceptable carriers suitable for use in such compositions are well known in the art of pharmacy. The compositions of the invention may contain 0.1-99% by weight of conjugated fatty acid. The compositions of the invention are generally prepared in unit dosage form. Preferably the unit dosage of conjugated fatty acid is from 1 mg to 1000 mg (more preferably from 100 mg to 750 mg). The excipients used in the preparation of these compositions are the excipients known in the art.

Further examples of product forms for the composition are food supplements, such as in the form of a soft gel or a hard capsule comprising an encapsulating material selected from the group consisting of gelatin, starch, modified starch, starch derivatives such as glucose, sucrose, lactose and fructose. The encapsulating material may optionally contain cross-linking or polymerizing agents, stabilizers, antioxidants, light absorbing agents for protecting light-sensitive fills, preservatives and the like. Preferably, the unit dosage of conjugated fatty acid in the food supplements is from 1 mg to 1000 mg (more preferably from 100 mg to 750 mg).

The following non-limiting examples illustrate the invention and do not limit its scope in any way. In the examples and throughout this specification, all percentages, parts and ratios are by weight unless indicated otherwise.

### EXAMPLES

### BRIEF DESCRIPTION OF THE DRAWINGS

FIG. 1 is a plot of Jackson Score against number of days for the patients treated with the placebo (upper line, black) and those treated with CLA (lower line, grey).

FIG. 2 shows the results of tests carried out in the morning and shows Total Symptom Score against number of days. Results for the group of people treated with CLA are shown on the left in grey (Series 1) and for people given the placebo are shown on the right in dark grey (Series 2).

FIG. 3 corresponds to FIG. 2 but relates to tests carried out in the afternoon.

FIG. 4 is a graph showing total symptom score and individual symptoms for the CLA group (light grey bars) and the placebo group (dark grey bars).

### EXAMPLES 1 AND 2

In Vivo Study

Protocol

45 human volunteers suffering from the common cold were used in the study. 21 subjects were administered conjugated linoleic acid (CLA) and the other 24 subjects were given a placebo.

The 21 subjects underwent pre-treatment with CLA at a level of 1.7 g/day for 4 weeks. 24 subjects underwent pre-treatment with placebo (HOSF; high oleic sunflower acids).

All subjects were inoculated at day 0 by intranasal exposure to human Rhinovirus (HRV) and the two groups were monitored daily for the next 5 days. The effects of the two types of treatment were determined by using the Jackson Score (validated in severity of symptoms) and the effects of the symptoms on its own on day 0-5.

Symptoms were assessed using a Jackson Score validated in severity from 0=absent to 3=very severe. The following symptoms were rated:

| | |
|---|---|
| Runny nose | Stuffiness |
| Sneezing | Sore throat |
| Cough | Headache |
| Malaise | Chilliness |

### EXAMPLE 1

Recovery After the Common Cold

The primary endpoint for the study was the frequency of clinical colds defined in accordance with the modified Jackson criteria. A subject will be considered to have a clinical cold if he/she has a cumulative symptom score of 6 or greater over the five days post-challenge (adjusted for any baseline symptom) and either reports runny nose on 3 post challenge days or responds “yes” to the question on day 5 post challenge whether he/she feels that he/she has had a cold during the previous 5 days.

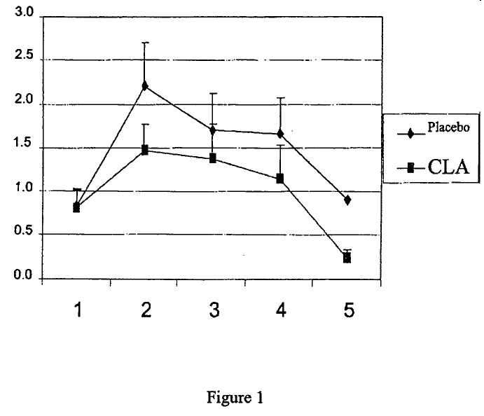

FIG. 1 is a plot of Jackson Score against number of days for the patients treated with the placebo (upper line, black) and those treated with CLA (lower line, grey).

The results show that at day 2 already, the severity of the common cold is lower in the CLA treated people. Given the fact that the difference already occurred at day 2 demonstrated the positive effect of CLA.

### EXAMPLE 2

Treatment of Symptoms

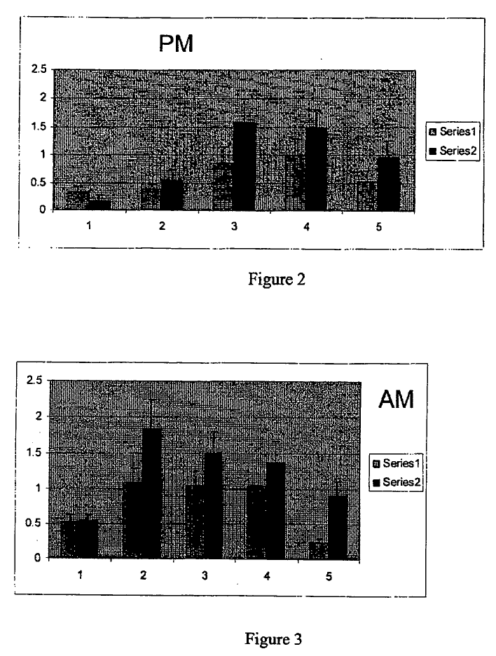

Total symptoms of the individuals were checked in the morning (am) and in the afternoon (pm). The results of the test in the morning are shown in FIG. 2 and the results of the test in the afternoon are shown in FIG. 3; both plots are of Total Symptom Score against number of days. In FIGS. 2 and 3, results for the group of people treated with CLA are shown on the left in grey (Series 1) and for people given the placebo are shown on the right in dark grey (Series 2).

The results showed that the total symptom score at day 2 until the end of the study is lower in the CLA treated people.

### EXAMPLE 3

Assessment of Total Symptoms Separately

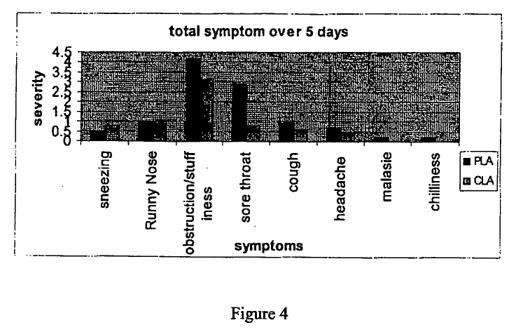

The symptoms assessed were sneezing, runny nose, obstruction, stuffiness, sore throat, cough, headache, malaise and chilliness. The total symptom score was determined by adding the scores for the symptoms during the 5 day period. FIG. 4 is a graph showing total symptom score and individual symptoms for the CLA group (light grey bars) and the placebo group (dark grey bars).

The results in FIG. 4 show that the severity of the symptoms assessed separately is very clearly lower in the CLA group (CLA) than in the placebo group (PLA).

### EXAMPLE 4

The following is an example of a filled gelatin capsule according to the invention. A composition comprising 60% by weight trans10, cis12 conjugated linoleic acid and 40% by weight cis9, trans11 linoleic acid is encapsulated into a gelatin capsule according to methods well-known in the art. The resulting encapsulated product contains 500 mg of the mixture of conjugated linoleic acid isomers and one tablet can be taken up to four times daily by an adult human.

1. A method of preventing or treating a common cold in a mammal, or of treating or ameliorating the symptoms of a common cold in a mammal, which comprises administering to said mammal an effective amount of one or more substances selected from conjugated fatty acids and derivatives thereof.

2. Method as claimed in claim 1, wherein said mammal is a human.

3. Method as claimed in claim 1, wherein said mammal is administered a composition comprising a conjugated fatty acid or a derivative thereof and wherein said composition is a pharmaceutical composition, a foodstuff or a food supplement.

4. Method as claimed in claim 1, wherein the conjugated fatty acid or derivative thereof is conjugated linoleic acid or a derivative thereof.

5. Method as claimed in claim 1, for reducing the recovery time after a common cold.

6. Method as claimed in claim 1, wherein the common cold is caused by a coronavirus or a rhinovirus.

7. Method as claimed in claim 1, wherein the amount of conjugated fatty acid or derivative thereof is from about 0.1 g to about 20 g of conjugated fatty acid or derivative thereof per day.

8. Method as claimed in claim 4, wherein the conjugated linoleic acid or derivative thereof comprises trans10, cis12 and cis9, trans11 isomers and the weight ratio of trans10, cis12 isomer to cis9, trans11 isomer is at least 1.2:1.

9. Method as claimed in claim 1, wherein said mammal is administered a composition comprising a conjugated fatty acid or a derivative thereof and wherein said composition is a foodstuff selected from the group consisting of margarines, fat continuous or water continuous or bicontinuous spreads, fat reduced spreads, confectionery products such as chocolate or chocolate coatings or chocolate fillings or bakery fillings, ice creams, ice cream coatings, ice cream inclusions, dressings, mayonnaises, cheeses, cream alternatives, dry soups, drinks, cereal bars, sauces, snack bars, dairy products, clinical nutrition products and infant formulations.

10. Method as claimed in claim 1, wherein said mammal is administered a composition comprising a conjugated fatty acid or a derivative thereof and wherein said composition is a pharmaceutical composition in the form of a tablet capsule, solution or emulsion.

11. Method as claimed in claim 1, wherein said mammal is administered a composition comprising a conjugated fatty acid or a derivative thereof and wherein said composition is a food supplement in the form of a soft gel or a hard capsule comprising an encapsulating material selected from the group consisting of gelatin, starch, modified starch, starch derivatives such as glucose, sucrose, lactose and fructose.

12. The use of a conjugated fatty acid or a derivative thereof in the manufacture of a composition for the prevention or treatment of the common cold, or the treatment or amelioration of symptoms of the common cold in a mammal.

13. Use as claimed in claim 12, wherein the mammal is a human.

14. Use as claimed in claim 12 or claim 13, wherein the composition is a pharmaceutical composition, a foodstuff or a food supplement.

15. Use as claimed in any one of claims 12 to 14, wherein the composition is for reducing the recovery time after a common cold.

16. Use as claimed in any one of claims 12 to 15, wherein the common cold is caused by a coronavirus or a rhinovirus.

17. Use as claimed in any one of claims 12 to 16, wherein the composition comprises an amount of conjugated fatty acid or derivative thereof such that the mammal receives a dose of 0.1 to 20 g of conjugated linoleic acid or derivative thereof per day.

18. Use as claimed in any one of claims 12 to 17, wherein the conjugated fatty acid or derivative thereof is conjugated linoleic acid or a derivative thereof.

19. Use as claimed in claim 18, wherein the conjugated linoleic acid or derivative thereof comprises trans10, cis12 and cis9, trans11 isomers and the weight ratio of trans10, cis12 isomer to cis9, trans11 isomer is at least 1.2:1.

20. Use as claimed in claim 18 or claim 19, wherein the conjugated linoleic acid or derivative thereof comprises at least 50% by weight of the trans10, cis12 isomer of conjugated linoleic acid.

21. Use as claimed in any one of claims 12 to 20, wherein the composition is a foodstuff selected from the group consisting of margarines, fat continuous or water continuous or bicontinuous spreads, fat reduced spreads, confectionery products such as chocolate or chocolate coatings or chocolate fillings or bakery fillings, ice creams, ice cream coatings, ice cream inclusions, dressings, mayonnaises, cheeses, cream alternatives, dry soups, drinks, cereal bars, sauces, snack bars, dairy products, clinical nutrition products and infant formulations.

22. Use as claimed in any one of claims 12 to 20, wherein the composition is a pharmaceutical composition in the form of a tablet, capsule, solution or emulsion.

23. Use as claimed in any one of claims 12 to 20, wherein the composition is a food supplement in the form of a soft gel or a hard capsule comprising an encapsulating material selected from the group consisting of gelatin, starch, modified starch, starch derivatives such as glucose, sucrose, lactose and fructose.
